(19) United States
(12) Patent Application Publication
(10) Pub. No.: US 2022/0365076 A1
(43) Pub. Date: Nov. 17, 2022

(54) MARKER AND SENSING SYSTEM USING THE SAME

(71) Applicants: KABUSHIKI KAISHA TOSHIBA, Tokyo (JP); TOSHIBA TEC KABUSHIKI KAISHA, Tokyo (JP)

(72) Inventors: Hiroki IWANAGA, Yokohama Kanagawa (JP); Ryozo AKIYAMA, Mishima Shizuoka (JP); Naoko FUTAMI, Mishima Shizuoka (JP)

(21) Appl. No.: 17/865,412

(22) Filed: Jul. 15, 2022

Related U.S. Application Data
(62) Division of application No. 16/596,336, filed on Oct. 8, 2019.
Application No. 16/596,336 is a continuation of application No. 15/366,146, filed on Dec. 1, 2016, now abandoned.

(30) Foreign Application Priority Data
Dec. 3, 2015 (JP) 2015-236964

(51) Int. Cl. G01N 33/52; G01N 21/82; G01N 21/64; G01N 33/02

(52) U.S. Cl. CPC G01N 33/52; G01N 21/82; G01N 21/6408; G01N 21/6428; G01N 33/02; G01N 2021/7786

(57) ABSTRACT

Certain embodiments provide a marker for detecting a detection target substance, including an assay reagent having a medium, and a composition containing a solvent or a solvent mixture, and a fluorescent coagulation factor that has a fluorescence property that changes as it coagulates in presence of the detection target substance. A difference of a hydrophobicity parameter LogP between the solvent and a part or the entirety of the detection target substance is equal to or smaller than 1.53.

### CROSS-REFERENCE TO RELATED APPLICATION

This application is a division of U.S. patent application Ser. No. 16/596,336, filed on Oct. 8, 2019, which is a continuation of U.S. patent application Ser. No. 15/366,146, filed on Dec. 1, 2016, now abandoned, which application is based upon and claims the benefit of priority from Japanese Patent Application No. 2015-236964, filed on Dec. 3, 2015, the entire contents of which are incorporated herein by reference.

### FIELD

Embodiments of the present invention relate to a marker capable of conveniently determining food freshness and a sensing system using the same.

### BACKGROUND

Amines generated from food decomposition (hereinafter, referred to as “biogenic amines” or simply “amines”) are produced through decarbonation of an amino acid and may cause a heath risk such as an allergic disease or food poisoning in fish, shellfish, or meat. Since the biogenic amine is generated even when edible food is processed or stored, the amount of the generated biogenic amines serves as an index indicating freshness of edible food. In recent years, a method of sensing the biogenic amine with high sensitivity and high selectivity is demanded in order to prevent an allergic disease or food poisoning and reduce food wastes.

As a method of sensing a biogenic amine, an instrumental analysis such as gas chromatography or liquid chromatography is known in the art. However, such an instrumental analysis requires a lot of time for pre-treatment performed before measurement, and a device for the instrumental analysis is to be carefully maintained and calibrated at all times. Therefore, the instrumental analysis is expensive.

Meanwhile, a simple and fast method for sensing a biogenic amine using a tetraphenylethene-based fluorescent substance is known in the art. In this sensing technique, the tetraphenylethene-based fluorescent substance is dissolved in a solution. In a first process, the biogenic amine is dissolved in a solution. In a second process, the tetraphenylethene-based fluorescent substance and the biogenic amine are reacted with each other to produce coagulations. It is known that, although a single particle of the tetraphenylethene-based fluorescent substance has a weak fluorescence intensity, coagulations of the tetraphenylethene-based fluorescent substance provide a stronger fluorescence intensity. Therefore, generation of a biogenic amine can be detected based on the increase of the fluorescence intensity. As a result, it is possible to sense deterioration of food freshness.

A lot of types of biogenic amines are generated through food decomposition, and they have different hydrophobicity. For this reason, while a type of the biogenic amine suitable for the solvent employed in the aforementioned sensing method is detected, another type of the biogenic amine having hydrophobicity significantly different from that of this type is not dissolved in the employed solvent and is not detected.

Since the type of the biogenic amine is different in each food, it is necessary to change the solvent depending on a type of target food if the aforementioned sensing method of the prior art is employed. This is disadvantageous.

### DESCRIPTION OF THE DRAWINGS

The accompanying drawings, which is incorporated in and constitute a part of this specification, illustrates an embodiment of the invention and together with the description, serve to explain the principles of the invention.

FIGS. 1A and 1B are a diagram illustrating a relationship of a hydrophobicity parameter LogP between a glycol-based solvent and a biogenic amine;

FIGS. 2A and 2B are diagrams illustrating an exemplary biogenic amine detection process for a maker according to Embodiment 1;

FIG. 3 is a schematic diagram illustrating a maker according to Embodiment 2;

FIG. 4 is a schematic diagram illustrating a maker according to Embodiment 3;

FIGS. 5A to 5C are diagrams illustrating a method of preparing a marker sample;

FIG. 6 is a diagram illustrating a method of evaluating the marker sample;

FIGS. 7A and 7B are images showing fluorescent states of the marker sample A;

FIGS. 8A and 8B are images showing fluorescent states of the marker sample B;

FIG. 9 is a fluorescence spectrum of a compound (1) immediately after adding spermidine;

FIG. 10 is a fluorescence spectrum of the compound (1) after adding spermidine and irradiating black light for fourteen hours; and

FIG. 11 is a fluorescence spectrum of the compound (1) after adding spermidine and retaining the compound (1) under the indoor light at the room temperature for thirteen days while it is opened to the atmosphere.

### DETAILED DESCRIPTION

Reference will now be made in detail to the present embodiment of the invention, an example of which is illustrated in the accompanying drawing.

A marker according to this embodiment is a marker for detecting a detection target substance. The marker has an assay reagent having a medium and a composition containing a solvent and fluorescent coagulation factor that has a fluorescence property that changes as it coagulates in presence of the detection target substance. The assay reagent is divided into two or more segments, and each of the assay reagent segments has different hydrophobicity for the solvent.

Embodiments of the present invention will now be described in detail with reference to the accompanying drawings.

According to this embodiment, the assay reagent has a fluorescent coagulation factor that has a fluorescence property that changes as it coagulates in presence of a biogenic amine serving as the detection target substance, a solvent for dissolving the fluorescent coagulation factor, and a medium for retaining the fluorescent coagulation factor and the solvent. The assay reagent is divided into two or more segments, and each of the assay reagent segments has different hydrophobicity for the solvent. Using the marker according to this embodiment, each assay reagent segment has different hydrophobicity for the solvent. Therefore, it is possible to detect many types of biogenic amines with high accuracy.

### (Biogenic Amine)

In general, if food is kept aside, a change occurs in odor, appearance, texture, taste, and the like as time elapses, and finally, the food becomes inedible. Such a decay of food is called deterioration, putrefaction, or spoilage, and is also typically described as “food is rotten.” The food deterioration occurs due to microorganisms, insects, autolysis, chemical reasons (oxidation of lipide or browning), or physical reasons (such as injury, crush, or damage). In many cases, food becomes inedible due to spoilage caused by proliferation of microorganisms (putrefactive bacteria), and this is called “decomposition” in a broad sense.

A process of generating harmful products or an unpleasant odor when food is decomposed due to a microorganism reaction generated in proteins of food is called “putrefaction.” In contrast, an inedible food state in which carbohydrate or fat and oil is decomposed due to a microorganism reaction, and a flavor of food is degraded may be distinguished from deterioration or spoilage in some cases. In addition, an amine component called various biogenic amines such as ammonia or trimethylamine is a main factor of a putrefactive odor.

For this reason, in order to analyze a decomposition level of food having rich protein such as meat or fish, it is useful to quantify this biogenic amine component. As a biogenic amine quantification analysis method, fast liquid chromatography is generally employed. However, a lot of time and cost are necessary in a complicated sample pretreatment, determination of a measurement time, and the like.

A nitrogen compound of food predominantly contains protein, and protein is hydrolyzed by a microorganism enzyme or a food enzyme to produce polypeptide, simple peptide, or an amino acid. In addition, the amino acid is decomposed through a deamination, transamination, or decarbonation reaction to produce a biogenic amine.

The biogenic amine produced from the amino acid includes, for example, 1,2-ethylenediamine, 1,3-propanediamine, 1,4-butanediamine, 1,5-pentanediamine, 1,6-hexanediamine, spermidine, spermine, histamine, tryptamine, or the like.

### (Fluorescent Coagulation Factor)

A fluorescent coagulation factor according to this embodiment refers to a fluorescent substance that coagulates or flocculates in presence of an amine to generate a change in a distribution or intensity of a fluorescence spectrum or an excitation spectrum or a change in a fluorescence property such as a fluorescence lifetime. As such a fluorescent coagulation factor, for example, coagulation-induced luminescent molecules discussed in JP 2012-51816 A may be employed. The coagulation-induced luminescent molecules do not emit fluorescent light even by irradiating excitation light while it is dissolved in a solvent. However, when they coagulate, fluorescent light is emitted. For example, the coagulation-induced luminescent molecules may be a tetra(aryl)ethene derivative as expressed in the following General Formula (I).

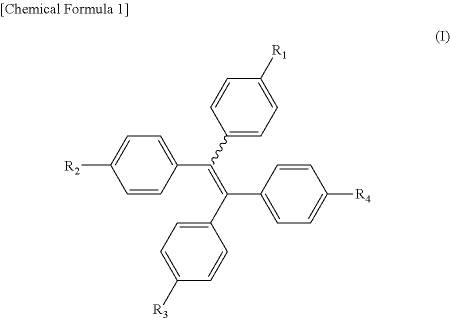

In this formula, the elements R₁, R₂, R₃, and R₄ are independent from each other and are selected from a group consisting of —COOM₁, —(CH₂)ₘ—COOM₂, —X—(CH₂)ₙ—COOM₃, —Y—(CH₂)ₒ—Z—(CH₂)ₚ—COOM₄ (where “M₁,” “M₂,” “M₃,” and “M₄” are independent from each other and denote a hydrogen atom or a cation, “X,” “Y,” and “Z” are independent from each other and denote “—O—,” “—NH—,” or “—S—,” and “m,” “n,” “o,” and “p” are independent from each other and denote integers between 1 to 6) , a hydrogen atom, a halogen atom, a hydroxyl group, a nitro group, a carbamoyl group, an alkyl group having a carbon number of 1 to 6, a haloalkyl group having a carbon number of 1 to 6, an alkenyl group having a carbon number of 2 to 6, a cycloalkyl group having a carbon number of 3 to 10, an alkyloxy group having a carbon number of 1 to 6, an acyl group having a carbon number of 2 to 6, an amino group, an alkylamino group having a carbon number of 1 to 6, an aryl group having a carbon number of 6 to 10, and a heteroaryl group having a carbon number of 5 to 10. Meanwhile, at least two of the elements R₁, R₂, R₃, and R₄ are independent from each other and are selected from a group consisting of —COOM₁, —(CH₂)ₘ—COOM₂, —X—(CH₂)ₙ—COOM₃, and —Y—(CH₂)ₒ—Z—(CH₂)ₚ—COOM₄ (where “M₁,” “M₂,” “M₃,” and “M₄” are independent from each other and denote a hydrogen atom or a cation, “X,” “Y,” and “Z” are independent from each other and denote “—O—,” “—NH—,” or “—S—, ” and “m,” “n,” “o,” and “p” are independent from each other and denote integers between 1 to 6).

Note that the cation of the Chemical Formula (1) described above may be either an organic or inorganic cation without a particular limitation. For example, the cation may include, for example, ammonium, alkali metal, alkali earth metal, pyridinium, or the like. In addition, if two or more cations exist in a single molecule, different cations may be included.

In the aforementioned General Formula (I), the tetra(aryl)ethene derivative coagulates because its solubility is degraded in a solution due to a hydrogen bond or an electrostatic interaction (hereinafter, also referred to as a “reaction”) between a carboxyl group and an amine in a molecule. If excitation light such as ultraviolet rays is irradiated onto this coagulating tetra (aryl) ethene derivative, it emits fluorescent light. According to this embodiment, a composition including the solvent and the fluorescent coagulation factor retained in a medium is created such that it has a concentration at which an unreacted fluorescent coagulation factor does not coagulate or precipitate, that is, no saturation occurs.

### (Solvent)

According to this embodiment, a solvent capable of dissolving the fluorescent coagulation factor is employed, and a difference of the hydrophobicity parameter LogP of all or a part of the biogenic amines as a detection target is set to “1.53” or smaller. If the difference of the hydrophobicity parameter LogP between the solvent and the biogenic amine as a detection target is greater than “1.53,” mutual solubility between the biogenic amine and the solvent is degraded, and the resulting coagulation becomes uneven. As a result, sensitivity of the mark may be degraded.

Note that the hydrophobicity parameter LogP refers to a distribution coefficient of a substance in water and 1-octanol and is calculated using a software program “Molecular Modeling Pro. Plus Version 7.0.4” produced by Norgwyn Montgomery Software, Inc.

According to this embodiment, since the marker is used to detect a time course of freshness deterioration, the solvent is preferably seldom volatilized and dried out under the atmosphere for a certain period of time. In addition, according to this embodiment, since the marker is added or neighbored to food, the solvent preferably has high safety for a human body. Furthermore, according to this embodiment, the solvent may be a solvent mixture obtained by mixing two or more types of solvents.

A glycol-based solvent having a high boiling point and low toxicity is preferably employed as the solvent according to this embodiment. Such a glycol-based solvent may include, for example, an ethylene glycol-based solvent such as polyethylene glycol monomethyl ether, diethylene glycol ethyl methyl ether, polyethylene glycol dimethyl ether, triethylene glycol butyl methyl ether, and diethylene glycol butyl methyl ether, or a propylene glycol-based solvent such as propylene glycol monomethyl ether, propylene glycol monobutyl ether, and propylene glycol dimethyl ether.

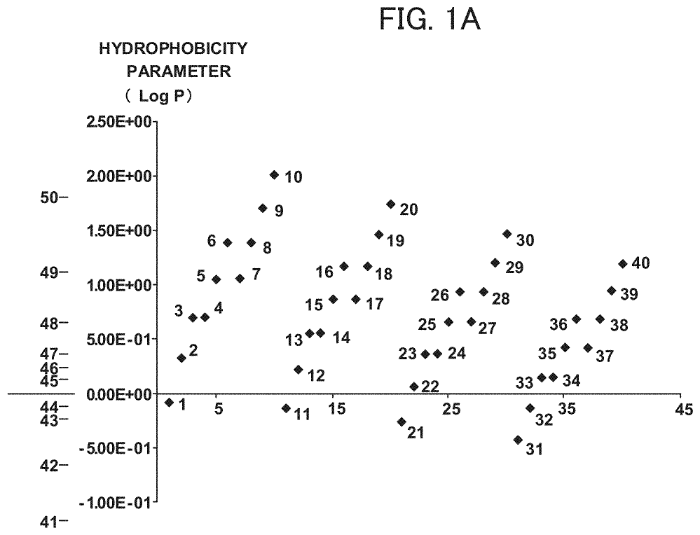

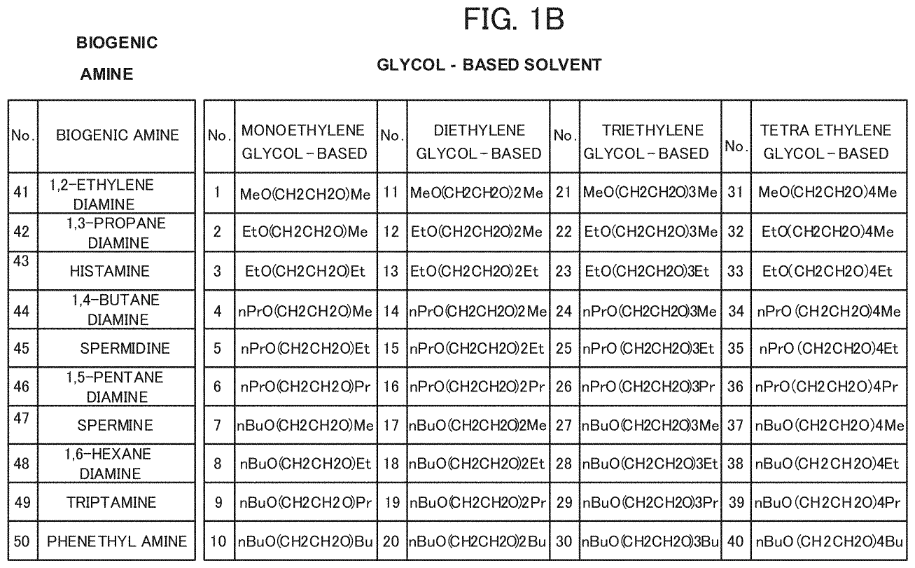

FIGS. 1A and 1B show an exemplary relationship or combination of the hydrophobicity parameter LogP between the glycol-based solvent and the biogenic amine.

As illustrated in FIGS. 1A and 1B, the hydrophobicity parameter LogP of the glycol-based solvent significantly depends on a repetition unit number of the terminal substituent group and the glycol unit. Specifically, the hydrophobicity parameter LogP increases as a total carbon number of the terminal substituent group increases. In addition, the hydrophobicity parameter LogP decreases as the repetition unit number of the glycol unit increases.

As the difference of the hydrophobicity parameter LogP between the solvent and the biogenic amine decreases, solubility of the biogenic amine increases. As the difference of the hydrophobicity parameter LogP between the solvent and the biogenic amine increases, solubility of the biogenic amine decreases. That is, mutual solubility is high between substances having the smaller difference of the hydrophobicity parameter LogP. If the difference of the hydrophobicity parameter LogP between the solvent and the detection target substance (decomposition substance) is small, this means that the solvent and the detection target substance are easily evenly mixed with each other.

Therefore, in the case of the glycol-based solvent illustrated in FIG. 1B, for example, the following assay reagents are preferably sequentially arranged on the basis of the relationship of the hydrophobicity parameter LogP between the biogenic amine and the solvent:

(1) an assay reagent having a total carbon number of the terminal substituent group set to “2” in the case of a monoethylene glycol-based solvent or a diethylene glycol-based solvent, having a total carbon number of the terminal substituent group set to “2 to 3” in the case of a triethylene glycol-based solvent, or having a total carbon number of the terminal substituent group set to “2 to 5” in the case of a tetraethylene glycol-based solvent;

(2) an assay reagent having a total carbon number of the terminal substituent group set to “3 to 4” in the case of a monoethylene glycol-based solvent, having a total carbon number of the terminal substituent group set to “3 to 6” in the case of a di glycol-based solvent, having a total carbon number of the terminal substituent group set to “3 to 6” in the case of a triethylene glycol-based solvent, or having a total carbon number of the terminal substituent group set to “3 to 7” in the case of a tetraethylene glycol-based solvent; and

(3) an assay reagent having a total carbon number of the terminal substituent group set to “5 to 8” in the case of a monoethylene glycol-based solvent, having a total carbon number of the terminal substituent group set to “6 to 8” in the case of a di glycol-based solvent, having a total carbon number of the terminal substituent group set to “7 to 8” in the case of a triethylene glycol-based solvent, or having a total carbon number of the terminal substituent group set to “7 to 8” in the case of a tetraethylene glycol-based solvent.

### (Medium)

According to this embodiment, any medium may be employed without a particular limitation as long as it can retain a composition (mixed solution) containing the fluorescent coagulation factor and the solvent described above. Considering retention of the composition (mixed solution), the medium preferably has a certain or higher level of porosity. For example, a porous substrate, a mesh structure, or the like may be employed.

Such a medium may include, for example, cellulose fiber, paper, fabric, a filter, a sponge, or the like. In particular, a membrane filter formed of a cellulose acetate material is preferably employed because it can increase the fluorescence intensity.

The medium is preferably selected from materials having a refractive index similar to that of the solvent according to this embodiment. If the employed medium has a refractive index similar to that of the solvent, the fluorescent light generated from the inside of the medium is not hindered. Therefore, it is possible to obtain a stronger fluorescence intensity and easily determine freshness.

### (Substrate)

A substrate capable of supporting the assay reagent may also be employed as necessary. The substrate preferably has a solvent resistance against the solvent used to dissolve the fluorescent coagulation factor. In addition, the substrate is preferably prohibited to emit fluorescent light by itself. Any material maybe employed in the substrate as long as it does not have a wavelength similar to a fluorescence wavelength that the fluorescent coagulation factor emits.

Such a member may include, for example, a Teflon (registered trademark) sheet, a polyimide sheet, a polyester film, a polyacetal sheet, a nylon sheet, a polycarbonate sheet, a polypropylene sheet, a polyethylene sheet, a PET film, a plastic sheet such as a vinyl chloride sheet, a glass plate, or the like.

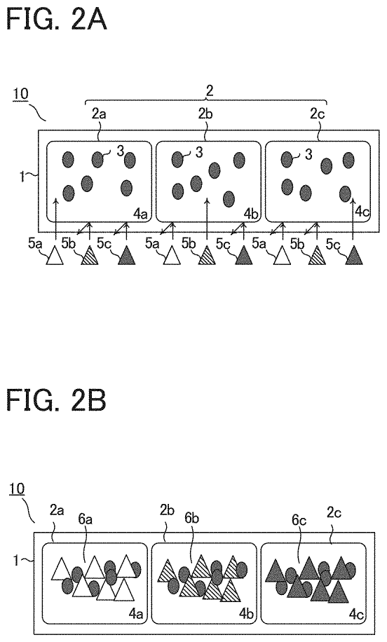

FIGS. 2A and 2B are diagrams illustrating an exemplary biogenic amine detection process using a marker according to this embodiment. As illustrated in FIG. 2A, the marker 10 according to this embodiment has an assay reagent 2 divided into assay reagent segments 2a, 2b, and 2c supported on a substrate 1. The assay reagent segments 2a, 2b, and 2c retain a fluorescent coagulation factor 3 and solvents 4a, 4b, and 4c, respectively, having different hydrophobicity. As illustrated in FIG. 2B, biogenic amines 5a, 5b, and 5c generated from food form coagulations 6a, 6b, and 6c, respectively, by coagulating with the fluorescent coagulation factor 3 in the assay reagent segments 2a, 2b, and 2c, respectively, where solvents 4a, 4b, and 4c, respectively, capable of easily dissolving the biogenic amines 5a, 5b, and 5c, respectively, are retained.

### (Determination Method)

The marker according to this embodiment detects a food freshness state by detecting a change of the fluorescence property caused by a reaction between the amine and the fluorescent coagulation factor retained on the medium as described above. Fluorescent light emitted by irradiating ultraviolet light onto the marker according to this embodiment from an UV light source unit is recognized using a light emission detection unit to determine a food freshness state.

A sensing system according to this embodiment includes the marker according to this embodiment, an ultraviolet (UV) light source unit that emits ultraviolet light onto the marker, and alight emission detection unit that detects an image pattern exhibited on the marker by irradiating ultraviolet rays. The detection target substance is detected on the basis of the image pattern exhibited on the marker by irradiating ultraviolet rays to determine a food freshness state. Here, the light emission detection unit refers to a visual inspection or an imaging device such as digital camera.

According to this embodiment, if the food freshness state is determined through a visual inspection as the light emission detection unit, the inspection is preferably performed in a dark place by avoiding a visible light atmosphere. In addition, using a fluorophotometer, it is possible to determine the freshness state with higher accuracy. Furthermore, by recognizing an image (image pattern) obtained using a charge-coupled device (CCD) image sensor or a complementary metal oxide semiconductor (CMOS) image sensor such as a digital camera, it is possible to perform the determination with higher accuracy.

Such an image electronically processed using a digital camera and the like can convert a weak fluorescent image into an image having a higher contrast. This method would be more useful when a user wants to determine a slight fluorescence intensity difference, that is, when a user wants to determine a very small difference of the amount of the biogenic amine. Furthermore, if a colorimetric function through image processing is provided in a camera-installed smart phone or the like, it is possible to determine freshness with an automatic determination function.

### (Another Embodiment of Marker)

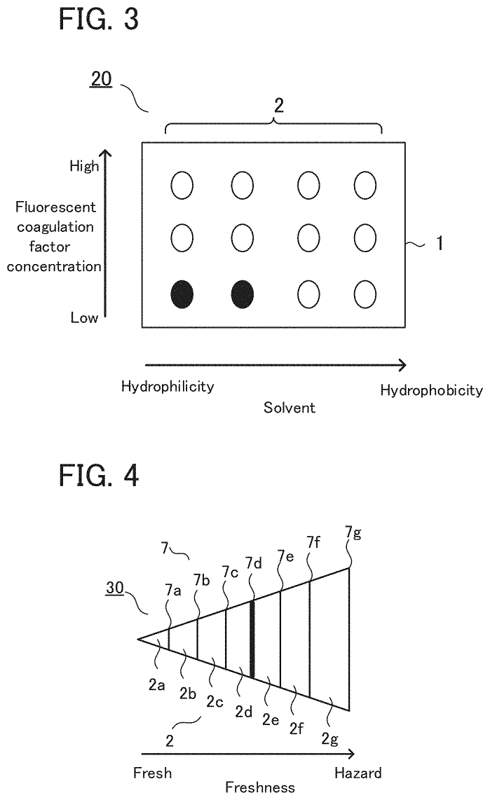

FIG. 3 illustrates a marker 20 according to Embodiment 2. As illustrated in FIG. 3, the marker 20 has a matrix arrangement including a plurality of assay reagents 2 two-dimensionally arranged on the substrate 1 by successively changing a hydrophobicity parameter LogP of the solvent and a concentration of the fluorescent coagulation factor. By successively changing the hydrophobicity parameter LogP of the solvent, it is possible to detect many types of biogenic amines. By successively changing the concentration of the fluorescent coagulation factor, it is possible to improve sensitivity for detecting the biogenic amine.

FIG. 4 is a diagram illustrating a schematic configuration of a marker 30 according to Embodiment 3. As illustrated in FIG. 4, the assay reagent 2 (the assay reagent segments 2a to 2g) of the marker 30 is arranged such that biogenic amines generated depending on a decomposition level can be sequentially detected. Along this arrangement, a freshness scale 7 (7a to 7g) is provided. As a result, it is possible to facilitate determination of freshness through a visual inspection. By inserting a bold line (indicated by reference numeral 7d in FIG. 4) positioned in a boundary between edibility and inedibility, it is possible to further facilitate determination of freshness through a visual inspection. Note that, although the assay reagent 2 has a triangular shape in FIG. 4, the shape of the assay reagent 2 is not particularly limited.

### EXAMPLES

Examples and comparative examples of the present invention will now be described in more detail. Note that the following examples are not intended to limit the present invention.

### Dependence on Biogenic Amines of Marker

### Creation of Fluorescent Liquid (Composition):

As the fluorescent coagulation factor, a compound (1) having a carboxyl group in positions of the elements R₁ and R₃ and a hydrogen atom in positions of the elements R₂ and R₄ in the general formula (I) is employed. The compound (1) is dissolved in the following solvent by 0.02 weight % to produce the fluorescent liquids A and B.

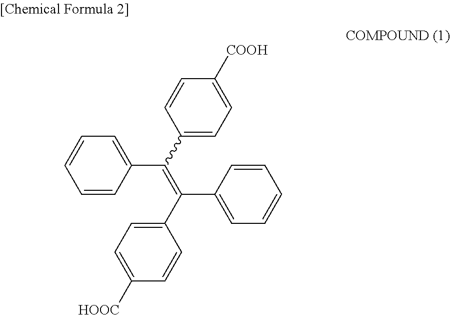

| | |
|---|---|
| Fluorescent Liquid A | |
| polyethylene glycol dimethylether | 99.98 wt % |
| (HISOLVE MPM, produced by TOHO | |
| Chemical Industry Co., Ltd) | |
| Compound (1) | 0.02 wt % |
| Fluorescent Liquid B | |
| triethylene glycol monobutyl ether | 99.98 wt % |
| (BTG, produced by Nippon Nyukazai Co., Ltd.) | |
| Compound (1) | 0.02 wt % |

### Manufacturing of Marker Sample:

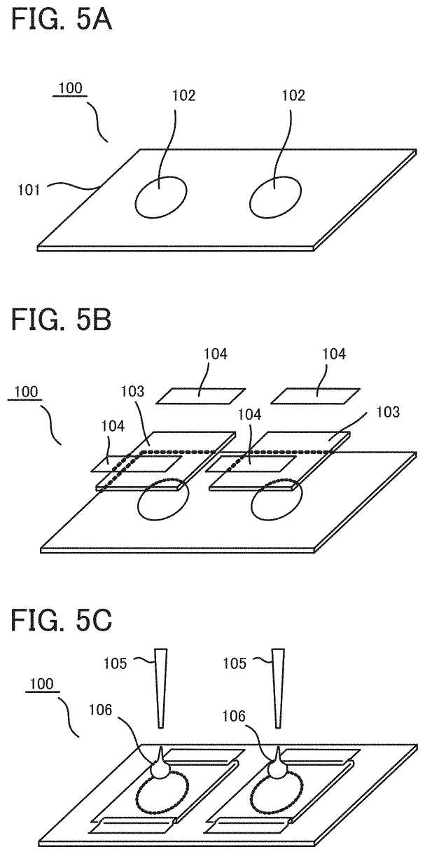

FIGS. 5A to 5C are diagrams illustrating a method of preparing an evaluation marker sample.

First, as illustrated in FIG. 5A, a pair of membrane filters 102 (CELLULOSE ACETATE C020A013A, produced by Adventech Co., Ltd.) were placed on a glass plate 101. Then, as illustrated in FIG. 5B, a screen gauze 103 (produced by MURAKAMI CO., LTD., with a gauze diameter of 34 μm and a thickness of 52 μm) was covered on each of the membrane filters 102, and its both ends were fixed using a polyimide adhesive tape 104.

Then, as illustrated in FIG. 5C, the fluorescent liquid 106 of 10 μL was dropped onto the membrane filter 102 above the screen gauze 103 using a pipette 105 to immerse the membrane filter 102 to form the assay reagent. As a result, an evaluation marker sample 100 was manufactured. The sample obtained by dropping the fluorescent liquid A as the fluorescent liquid 106 was selected as a marker sample A, and the sample obtained by dropping the fluorescent liquid B was selected as a marker sample B.

### Evaluation of Marker Sample:

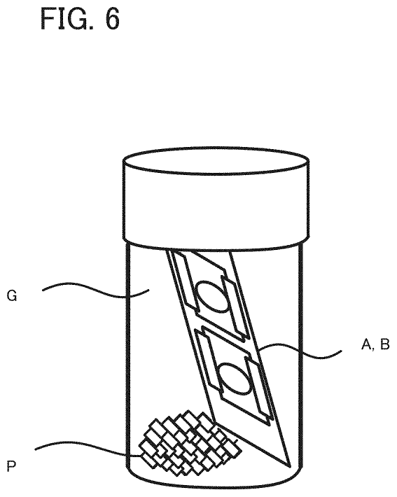

As illustrated in FIG. 6, each of the marker samples A and B manufactured as described above was inserted into a lid-covered glass container G with a raw food material P (fish paste) while they did not come into contact with the assay reagent. After the marker samples were inserted, a lid was installed, and they were stored under a room temperature atmosphere. Then, the fluorescent states of the marker samples were observed in the time course.

The fluorescent state was observed by picking out the marker samples from the glass container G and irradiating ultraviolet rays under an appropriate dark room state. Their fluorescent states were captured using a digital camera. The irradiation of ultraviolet rays was performed using a compact black light source.

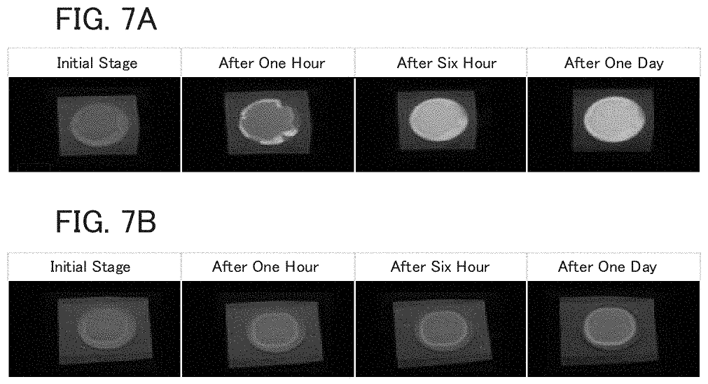

FIGS. 7A and 7B are images obtained by capturing the fluorescent state of the marker sample A using the fluorescent liquid A at an initial stage, after one hour, after six hours, and after one day. FIG. 7A shows images of the marker sample A placed in the glass container G with a raw food material P (fish paste). FIG. 7B shows images of the marker sample A placed in the empty glass container G without a food material P.

From the images of FIG. 7A, it is recognized that the intensity of the fluorescent light of the marker sample A increases as time elapses. In contrast, as recognized from the images of FIG. 7B, the marker sample A placed in the empty glass container has nearly no change in the fluorescence intensity.

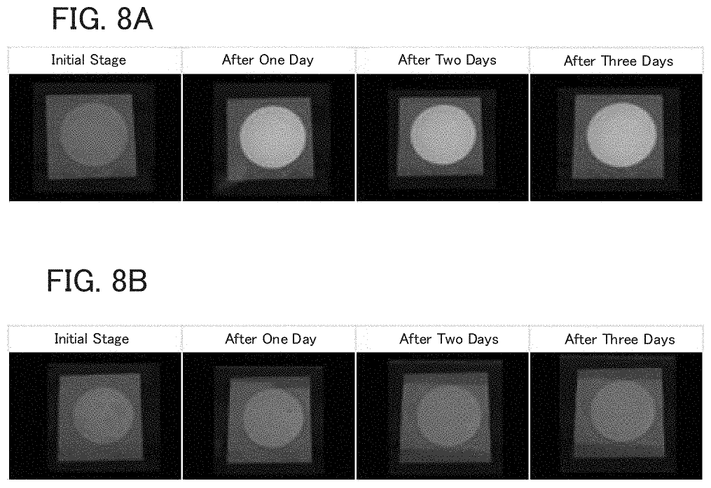

FIGS. 8A and 8B are images obtained by capturing the fluorescent state of the marker sample B using the fluorescent liquid B at an initial stage, after one day, after two days, and after three days. FIG. 8A shows images of the marker sample B placed in the glass container G with a raw food material (fish paste), and FIG. 8B shows images of the marker sample B placed in the empty glass container G without a food material.

It is recognized that a change of the fluorescence intensity of the marker sample B is insignificant relative to the marker sample A of the fluorescent liquid Ain both the glass container with a raw food material P (fish paste) and the empty glass container. In addition, even in the fluorescent state after three days, there is no significant increase of the fluorescence intensity.

That is, a high sensitive marker for recognizing a change of the freshness state in the food material (fish paste) used in this test is the marker sample A of the fluorescent liquid Ain which polyethylene glycol dimethyl ether is employed as a solvent.

As a result of the evaluation described above, it is recognized that it is necessary to create a fluorescent liquid suitable for the corresponding food material in order to prepare a marker suitable for various food materials.

### Dependence on Solvent in Fluorescence Spectrum

Dependence on the solvent in the fluorescence spectrum was evaluated for the compound (1) by selecting spermidine of a biogenic amine as a detection target substance. Various parameters of the used solvent and the spermidine are shown in Table 1. The hydrophobicity parameter LogP was calculated using Molecular Modeling Pro. Plus Version 7.0.4, produced by Norgwyn Montgomery Software, Inc. First, the structure was optimized using a molecular dynamics calculation (MM2), and the hydrophobicity parameter LogP was calculated using a three-dimensional structure activity relationship (three-D QSAR terms).

TABLE 1

| | | molecular | boiling point | viscosity | Log P | |
|---|---|---|---|---|---|---|
| No. | solvent | weight | (° C.) | (mP.s 20° C.) | (calculated value) | Δ Log P*⁵ |
| 1 | nBu—O—(CH₂CH₂O)₃—Me^{*1} | 220 | 261 | 2.9 | 0.66 | 0.44 |
| 2 | nBu—O—(CH₂CH₂O)₂—nBu^{*2} | 218 | 256 | 2.4 | 1.75 | 1.53 |
| 3 | Me—O—(CH₂CH₂O)₃—Me^{*3} | 178 | 216 | 2.2 | −0.25 | 0.47 |
| 4 | nBu—O—(CH₂CH₂O)₂—H^{*4} | 162 | 230 | 6.6 (H-bond) | 0.56 | 0.34 |

| | | | | |
|---|---|---|---|---|
| biogenic amine | 145 | 128 | 0.22 | — |
| (detection target substance) | | | | |
| spermidine | | | | |

[Remarks]
^{*1}triethylene glycol butyl methyl ether
^{*2}diethylene glycol dibutyl ether
^{*3}triethylene glycol dimethyl ether
^{*4}diethylene glycol monobutyl ether
^{*5}difference of LogP between solvent and spermidine

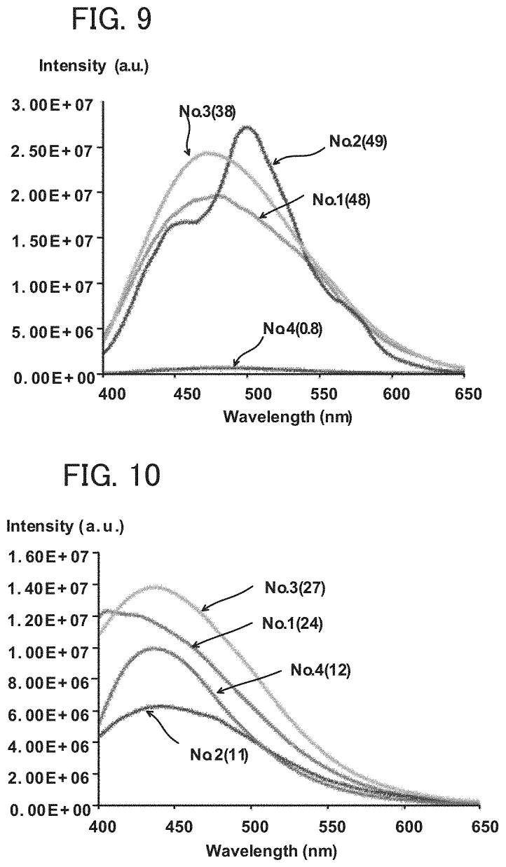

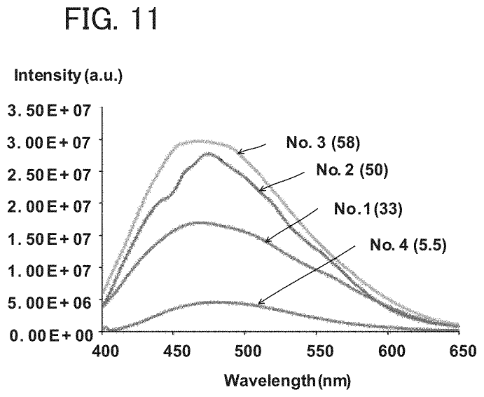

Fluorescence spectra obtained by adding an eight molar equivalent (500 μmol) of spermidine to each solvent of the compound (1) are illustrated in FIGS. 9 to 11. FIG. 9 illustrates a fluorescence spectrum obtained immediately after adding spermidine, and FIG. 10 illustrates a fluorescence spectrum obtained by adding spermidine and irradiating black light for fourteen hours. In addition, FIG. 11 illustrates a fluorescence spectrum obtained by adding spermidine and placing the compound (1) under the indoor light at the room temperature for thirteen days while it is opened to the atmosphere. In FIGS. 9 to 11, the numbers in the parentheses refer to magnification ratios of the fluorescence intensity with respect to the fluorescence intensity at the maximum wavelength when only the compound (1) is contained.

From the fluorescence spectra illustrated in FIGS. 9 to 11, it is recognized that the fluorescence intensity remarkably increases immediately after adding spermidine, excluding diethylene glycol monobutyl ether (No. 4). In addition, from the fluorescence spectra, it is also recognized that a long time is necessary until coagulation and fluorescent light are observed in the diethylene glycol monobutyl ether (No. 4). Such a solvent can be used to create a solvent mixture obtained by mixing a solvent with other solvents in order to improve coagulation stability.

As a comparative example in which spermidine is used as a detection target substance, a fluorescence spectrum of the compound (1) obtained by using dibutyl maleate having a hydrophobicity parameter LogP of 2.13 as the solvent (ΔLogP=1.91) was evaluated. As a result, the fluorescence intensity was remarkably reduced by adding spermidine and irradiating black light for fourteen hours.

However, there was no remarkable reduction in the fluorescence intensity when phenethylamine (LogP=1.84, ΔLogP with dibutyl maleate <0.29) having a hydrophobicity parameter LogP greater than that of the spermidine is used as a detection target substance.

From the evaluation result described above, if a difference of the hydrophobicity parameter LogP between the solvent used in the assay reagent of the marker and the detection target substance is greater than 1.53, the detection target substance is not dispersed in a molecular state in the solvent, but forms a minute droplet. As a result, it is conceived that the coagulation formed from the detection target substance and the fluorescent coagulation factor becomes uneven, and the retention of the fluorescence intensity is degraded.

In contrast, if the difference of the hydrophobicity parameter LogP is equal to or smaller than 1.53, an even coagulation network between the fluorescent coagulation factor and the detection target substance is easily formed, and such a coagulation has excellent durability. Meanwhile, if a solvent has a terminal “—OH” group as in the solvent of No. 4, it forms a hydrogen bond with a “—COOH” group of the fluorescent substance. Therefore, it is conceived that an interaction with a decomposed substance is limited. Such a solvent is advantageously added to a part of a solvent mixture in order to adjust sensitivity of the marker (necessary to adjust sensitivity depending on food). However, if the entire solvent is the No. 4 type, a coagulation rate is reduced.

### Fluorescence Lifetime

A fluorescence lifetime was evaluated immediately after solely adding the compound (1) to the solvents No. to 4, immediately after adding spermidine, after irradiating black light for ten hours, and after irradiating black light for fourteen hours. In addition, as a durability test, a fluorescence lifetime was evaluated after the sample is kept aside for thirteen days under the room light at the room temperature while it is opened to the atmosphere. The result of evaluation is shown in Table 2. Note that the fluorescence lifetime of Table 2 is obtained by approximating the attenuation function of the fluorescence into two types of expotential functions, and the numbers in the parenthesis refer to χ2 coefficients.

TABLE 2

| | | | compound (1) + spermidine | | | |
|---|---|---|---|---|---|---|
| | | | immediately | | | keeping at room |
| No. | solvent | compound (1) | after addition | UV 10 h | UV 14 h | temperature for 13 days |
| 1 | nBu—O—(CH₂CH₂O)₃—Me | 3.6 ns (43%) | 3.8 ns (33%) | 4.0 ns (26%) | 3.8 ns (26%) | 4.2 ns (32%) |
| | | 11.1 ns (57%) | 7.4 ns (67%) | 14.0 ns (74%) | 13.9 ns (77%) | 7.6 ns (68%) |
| | | (1.22) | (1.26) | (1.49) | (1.33) | (1.01) |
| 2 | nBu—O—(CH₂CH₂O)₂—nBu | 0.92 ns (8%) | 3.4 ns (28%) | 3.1 ns (%) | 3.4 ns (34%) | 3.8 ns (34%) |
| | | 8.2 ns (92%) | 6.7 ns (72%) | 9.8 ns (%) | 11.0 ns (64%) | 7.1 ns (68%) |
| | | (1.27) | (0.99) | (1.26) | (1.1) | (1.57) |
| 3 | Me—O—(CH₂CH₂O)₃—Me | 0.93 ns (6%) | 3.0 ns (36%) | 4.0 ns (33%) | 3.0 ns (34%) | 3.9 ns (34%) |
| | | 8.7 ns (94%) | 6.4 ns (64%) | 12.8 ns (67%) | 9.0 ns (66%) | 6.7 ns (66%) |
| | | (1.19) | (1.09) | (1.4) | (1.35) | (1.01) |
| 4 | nBu—O—(CH₂CH₂O)₂—H | 0.96 ns (7%) | 2.7 ns (15%) | 6.5 ns (35%) | 4.4 ns (29%) | 4.0 ns (31%) |
| | | 9.2 ns (93%) | 9.3 ns (85%) | 20.0 ns (65%) | 11.1 ns (71%) | 9.8 ns (69%) |
| | | | (1.01) | (1.69) | (1.31) | (1.17) |

Table 2 shows a change of the fluorescence lifetime of the compound (1) by adding spermidine as a detection target substance (decomposition substance) and a change of the fluorescence lifetime as a result of the durability test.

As shown in Table 2, in the cases of Nos. 1, 2, and 3 in which the fluorescence intensity increases by adding spermidine, the main fluorescence lifetime is reduced by adding spermidine. Meanwhile, in the case of No. 4 in which the fluorescence intensity does not increase, the main fluorescence lifetime is not reduced. This is because an interaction between the compound (1) and the spermidine increases, that is, because a coagulation is generated.

Although the fluorescence intensity is reduced by irradiating UV light, the fluorescence lifetime increases accordingly. It is conceived that this is because an interaction between the compound (1) and the spermidine is weakened. Meanwhile, the rightmost fluorescence lifetime in which the fluorescence intensity is not reduced even through the durability test does not increase. As a result, it is possible to know that this fluorescence lifetime can be used in freshness determination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions the accompanying claims and their equivalents are intended to cover such forms or modifications as would fall within the scope and spirit of the inventions.

What is claimed is:

1. A sensing system, comprising:
a marker for detecting a target substance, comprising an assay reagent including:
a composition containing a fluorescent material and a solvent or a solvent mixture, the fluorescent material having a fluorescence property that changes with coagulation in the presence of a target substance, the fluorescent material being soluble in solvent or the solvent mixture, and
a medium containing the composition, the medium being divided into divided medium portions forming assay reagent segments, each of the divided medium portions having a solvent or solvent mixture with a different hydrophobicity;
an ultraviolet light source to irradiate the marker with ultraviolet light; and
a detector to detect fluorescent light emitted from the marker.

2. The sensing system according to claim 1, wherein
coagulated fluorescent material emits fluorescent light upon irradiation with the ultraviolet light, and the marker exhibits an image pattern, and
the detector detects the presence of the target substance on the basis of the exhibited image pattern.

3. The sensing system according to claim 1, wherein the assay reagent detects a predetermined target substance by a difference an intensity of a fluorescence spectrum of the fluorescent material, a shape of a fluorescence spectrum of the fluorescent material, or a fluorescence lifetime of the fluorescent material.

4. The sensing system according to claim 1, wherein the fluorescent material is selected from tetra(aryl)ethenes.

5. The sensing system according to claim 1, wherein the solvent is a glycol-based solvent.

6. The sensing system according to claim 1, wherein the assay reagent segments are arranged in a two-dimensional matrix in which a solvent hydrophobicity parameter and a concentration of the fluorescent material are varied in a step-wise manner across the matrix.

7. The sensing system according to claim 1, wherein
each assay reagent segment has a different sensitivity to a predetermined target substance, the assay reagent segments are arranged in a sequence corresponding to a food decomposition level, and
a successive scale is provided along the sequence of assay reagent segments to indicate a food freshness level.

8. The sensing system according to claim 1, wherein a difference of a hydrophobicity parameter LogP between the solvent or the solvent mixture and a predetermined target substance is equal to or less than 1.53.
